(19) United States
(12) Patent Application Publication
(10) Pub. No.: US 2011/0257369 A1
(43) Pub. Date: Oct. 20, 2011

(54) METHODS FOR REMOVING NICOTINE AND OTHER ALKALOIDS FROM SOLUBLE LEAF PROTEINS IN SOLANACEOUS AND OTHER PLANT SPECIES

(75) Inventors: Yangming Martin Lo, Ashton, MD (US); Hong Fu, Fujian Province (JP)

(73) Assignee: UNIVERSITY OF MARYLAND, COLLEGE PARK, MD (US)

(21) Appl. No.: 13/124,798

(22) PCT Filed: Oct. 19, 2009
(86) PCT No.: PCT/US09/61191

§ 371 Date: Jul. 11, 2011

Related U.S. Application Data
(60) Provisional application No. 61/106,429, filed on Oct. 17, 2008.

(51) Int. Cl. C07K 1/14; C07K 1/34

(52) U.S. Cl. 530/370

(57) ABSTRACT

Described herein is a process for removing nicotine and other alkaloids from plant leaf proteins. The plant leaf proteins may be derived from tobacco }Nicotiana tabacum) and other solanaceous and toehr green leaf plants, both non-transgenic and transgenic. The present invention provides efficient techniques for removing nicotine and toehr alkaloids from solanaceous plant-derived leaf proteins to non-detectable levels. Significantly, use of the most preferred method does not substantially reduce leaf protein recovery. Application of these techniques could make leaf proteins derived from solanaceous species suitable for human and animal use consumption.

### GOVERNMENT INTERESTS

This invention was made with U.S. government support under USDA-CSREES Award No. 2006-34467-17102. The government has certain rights in the invention.

### FIELD OF THE INVENTION

This invention relates to the removal of nicotine and other alkaloids from plant leaf proteins.

### BACKGROUND OF THE INVENTION

Leaf proteins are potentially the cheapest and most abundant source of protein in the world (Pirie, 1987). (Full reference citations of all references mentioned herein are included at the end of the specification.) They are also highly nutritious and have many desirable functional characteristics which could make them useful in both food and industrial products.

One of the most promising sources of leaf proteins is tobacco (Nicotiana tabacum), based on its capacity to produce large volumes per acre of leaf protein. Tobacco and other solanaceous plants are a potentially rich source of plant leaf proteins. However, solanaceous plants contain nicotine and other harmful alkaloids, which could limit the uses of leaf proteins.

The majority of prior research involving nicotine removal has focused on the removal of nicotine from tobacco itself or from cigarette smoke, rather than from tobacco-derived leaf proteins. Many of these procedures involved use of solvents and particularly expensive supercritical solvents.

Roselius et al. (1979) developed a process for selectively extracting nicotine from tobacco by exposing the tobacco to an extracting solvent in either liquid or gaseous state at high pressures. Aroma-generating substances are first removed from the tobacco by conducting this extraction while the tobacco is dry. The tobacco is then moistened, and the nicotine is removed on further contact. The aroma-generating substances are then recombined with the nicotine-free tobacco, resulting in a tobacco product which contains aroma-generating substances but does not contain nicotine. This process was utilized to remove nicotine from tobacco but not from leaf protein.

Prasad et al. (1996) disclosed a process for removing nicotine from tobacco, which involved feeding an essentially nicotine-free supercritical solvent into an extraction flow system containing tobacco and then withdrawing a nicotine-rich solvent from the other end of the system. This system differed from the claimed invention in that it utilized supercritical solvents to extract nicotine, and that it was only demonstrated for use with nicotine and not specifically for leaf proteins.

Nauryzbaev et al. (2008) developed a technique for removing nicotine from tobacco raw material using low-boiling organic solvents, such as petroleum ether, chloroform or methylenechloride at a raw-material:solvent weight ratio of about 1:3. The solvent extraction of nicotine is carried out for about five hours in vapor phase and is then followed by solvent extraction. This method is for removing nicotine from tobacco raw material, and there is no evidence presented that it would be suitable for use with leaf proteins. Furthermore, the use of organic solvents would likely denature the leaf proteins and present environmental compliance issues not present in the claimed invention.

Casey (1978) developed a method for removing nicotine from tobacco, which involved rapidly drying an aqueous dispersion comprising particulate, with the dispersion having an alkaline pH which volatilizes free nicotine. Casey reported that multiple rewetting and redrying of tobacco using this procedure could achieve nicotine reduction of 70%. Casey's method was for removing nicotine from tobacco, rather than from leaf proteins, and significant amounts of nicotine would remain in the tobacco even after multiple repetitions of this procedure.

Browne et al. (1995) reported that nicotine could be removed from tobacco smoke through use of compounds containing a metal having a valence of +2. However, this technique was not demonstrated or claimed to work for leaf proteins or even from raw tobacco.

Wildman et al. (1981) reported a method for producing a deproteinized tobacco, which also removed nicotine along with the protein from the tobacco product. Wildman's method involved the separation of a yellow, water-soluble pigmented material from insoluble green pigmented material. The yellow materials would then be oxidized by adding ammonium hydroxide or other volatile bases which have boiling points similar to or below that of water or by bubbling ammonia gas through the aqueous solution containing the yellow pigmented material to adjust the pH to about 10.5. Bubbling air or oxygen through the solution would then cause the solution to turn brown. The brown solution would then be heated to remove volatile bases including nicotine. Following removal of the nicotine, the remaining brown solution would be sprayed back onto the solid tobacco material. This method was not intended to remove nicotine from leaf proteins. Rather, it was intended to remove both nicotine and protein from the tobacco, resulting in a deproteinized, low-nicotine tobacco product intended for use in cigarettes.

To the extent that research has focused specifically on nicotine removal from tobacco-derived proteins, research has focused on use of supercritical fluid extraction (SFE). Fantozzi et al. (1993) evaluated the use of supercritical CO₂ extraction of nicotine from tobacco leaf protein. They reported that it was possible to remove 99% of the nicotine from fraction-1 leaf protein (also known as ribulose-1,5-bisphosphate (RUBP) carboxylase/oxygenase or “rubisco”) on a pilot scale using supercritical CO₂. They also reported that nicotine extraction yield increases with increases in sample moisture. However, supercritical fluid extraction is a costly process which could render leaf proteins prohibitively expensive in many or most commercial applications.

As noted earlier, leaf proteins represent a potentially important new source of proteins for both food and industrial applications. Tobacco and other solonaceous plants are potentially important sources of leaf proteins.

However, nicotine is endogenously produced in tobacco plants and makes up approximately 98% of the total alkaloids in tobacco. Nicotine can be rapidly absorbed in humans and is very toxic at higher doses, and is highly addictive. In addition to nicotine, tobacco contains other alkaloids which pose health risks such as nornicotine, anabatine, anabasine and myosimine.

During the process of recovering tobacco leaf proteins, nicotine can enter into and contaminate the leaf protein products if no further and pertinent procedures are adopted, because of nicotine's solubility in water and common solvents. Therefore, it is critical to find a method to process tobacco leaf proteins in which nicotine and other harmful alkaloids are removed. The potential commercial applications of leaf protein from solonaceous plants may be severely limited unless nicotine and other alkaloids can be removed.

Tobacco shows great promise as a source of leaf proteins. Leaf proteins derived from tobacco have a nutritional content comparable to milk, and are considered hypoallergenic. The proteins also have excellent gelling, foaming, binding and emulsifying characteristics. They are also odorless and tasteless, which permits them to be added to products without imparting undesirable food or industrial characteristics.

Given these characteristics, tobacco leaf proteins could potentially be used in a wide range of products for human or animal consumption. However, it is unlikely that these uses will become viable unless the nicotine and other harmful alkaloids can be removed from the proteins first.

There remains a need in the art for methods for removal of nicotine and other alkaloids from plant leaf proteins. The inventors have developed efficient techniques for removing nicotine and other alkaloids from solanaceous plant-derived leaf proteins. These techniques have been demonstrated to remove alkaloids from leaf protein down to non-detectable levels. Significantly, use of the most preferred method does not substantially reduce leaf protein recovery. Application of these techniques could make leaf proteins derived from solanaceous species suitable for widespread human and animal use consumption.

### SUMMARY OF THE INVENTION

Using high-performance liquid chromatography to analyze nicotine content, three different methods of removal nicotine from tobacco protein were developed and evaluated: (1) removal of nicotine with acetone (2) ultrafiltration; and (3) three steps of protein precipitation with phosphoric acid.

We found that both the acetone and phosphoric acid treatments removed nicotine to non-detectable levels. The phosphoric acid treatment, however, provided a substantially higher leaf protein recovery than the acetone. Specifically, we found that by adding 85% phosphoric acid to the water-soluble protein until we obtained a solution pH of 3.5, permitting the leaf protein to precipitate out of the solution, removing the nicotine-containing supernatant, and dissolving the precipitate in buffer and then repeating this treatment one or more times, it was possible to obtain water-soluble protein recovery of 94.56% with a nicotine-free product. In conclusion, a nicotine-free protein from leafy plants was accomplished.

In some embodiments, the present invention provides a method for removing alkaloids from water-soluble leaf proteins. Such methods may comprise applying an acid to a solution containing water-soluble leaf proteins; separating the alkaloids from the water-soluble leaf proteins; and collecting the water-soluble leaf proteins. In one embodiment, acid is added until the solution has a pH of from about 2.5 to about 5.0, for example, 3.5. Any acid known to those skilled in the art may be used to adjust the pH of the solution. Suitable examples include, but are not limited to, phosphoric acid, sulfuric acid, citric acid, nitric acid, tartaric acid, malic acid, lactic acid, malonic acid, succinic acid, acetic acid, glutamic acid, glucaric acid, itaconic acid, levulinic acid, fumaric acid, aspartic acid, propionic acid, monopotassium citrate, or furandicarboxylic acid. In one embodiment, the acid comprises phosphoric acid, for example, 85% phosphoric acid.

As the solution reaches an optimal pH level, soluble leaf protein will precipitate out of the solution, resulting in a separation of the leaf protein from the soluble nicotine and nicotine salts. It is possible to passively permit the proteins to precipitate out of the solution. Alternatively, separation may be accelerated using any technique known to those skilled in the art. In some embodiments, separating may comprise centrifugation and/or filtration. One suitable set of conditions for centrifugation is to centrifuge at 12,000 g for at least one minute and not more than 20 minutes. Following separation, the soluble leaf proteins are collected in the precipitate (or retentate, if filtration is used).

After the water-soluble leaf proteins are collected, the precipitate may be dissolved (e.g., in a buffer solution) the separation process may be repeated one or more times to achieve a desired level of alkaloids. If the separation process is to be repeated, it is contemplated that the leaf proteins may be dissolved in a buffering solution comprising one or more of, for example, phosphate buffers, hydrochloric acid buffers, boric acid buffers, sodium hydroxide buffers, citric acid or citrate buffers, or carbonate buffers. It is desirable to adjust the pH of this solution to a range within the buffering region of the selected buffer and at a pH which maximizes the solubility of the leaf proteins. The buffer solution may be added in a ratio of from 1:10 to 10:1 v/v compared with the original volume of the leaf protein solution prior to the addition of acid as described above. (For example, if the leaf protein solution initially contained 100 ml by volume prior to the acid pH adjustment and separation steps, then between 10 ml and 1 liter of buffer solution should be added to resolubilize the precipitated leaf proteins). In one embodiment of this invention, a .067 M solution of Na₂HPO₄-KH₂PO₄ at a pH of approximately 7.7 at a ratio of buffer to original leaf solution of 1:1 may be utilized.

Following resolubilization of the leaf protein solution, the steps of applying an acid to the buffer solution containing water-soluble leaf proteins; separating the alkaloids from the water-soluble leaf proteins; and collecting the water-soluble leaf proteins may be repeated one or more times. This process may be repeated, for example, 2, 3, 4, or 5 times until a desired level of alkaloids is achieved.

### BRIEF DESCRIPTION OF THE DRAWINGS

FIG. 1 is a flow chart showing a purification scheme in accordance with the present invention.

### DETAILED DESCRIPTION OF INVENTION

The principles, preferred embodiments and modes of operation of the present invention will be described hereunder. The invention which is intended to be protected herein should not, however, be construed as limited to the particular forms disclosed, as these are to be regarded as illustrative rather than restrictive. Variations and changes may be made by those skilled in the art without departing from the spirit of the present invention. Accordingly, the examples, descriptions, and best mode of carrying out the invention given below should be considered exemplary in nature and not as limiting to the scope and spirit of the invention as set forth in the claims.

Using the methods of this invention, the inventors have been able to completely remove nicotine and other alkaloids from tobacco down to non-detectable levels. The methods described herein offer several advantages over other known methods of removing nicotine, the majority of which have not been demonstrated to effectively remove nicotine from water-soluble leaf proteins (but rather only from tobacco or tobacco smoke). First, the claimed invention in its most preferred application uses GRAS materials, principally phosphoric acid, to remove nicotine. Second, it does not involve expensive processes such as use of supercritical solvents. Finally, the methods of this invention have been shown to result in minimal protein loss.

The claimed invention may comprise one or more of the following steps: (1) applying an acid to a solution containing water-soluble leaf proteins; (2) causing or permitting the water-soluble leaf proteins to separate from the nicotine and other alkaloids; (3) removing one or more alkaloids (e.g., nicotine); and (4) collecting the water-soluble leaf proteins. In some embodiments, the present invention provides a method of isolating water-soluble leaf proteins comprising:(1) applying an acid to a solution containing water-soluble leaf proteins; (2) causing or permitting the water-soluble leaf proteins to separate from the nicotine and other alkaloids; (3) removing one or more alkaloids (e.g., nicotine); and (4) collecting the water-soluble leaf proteins.

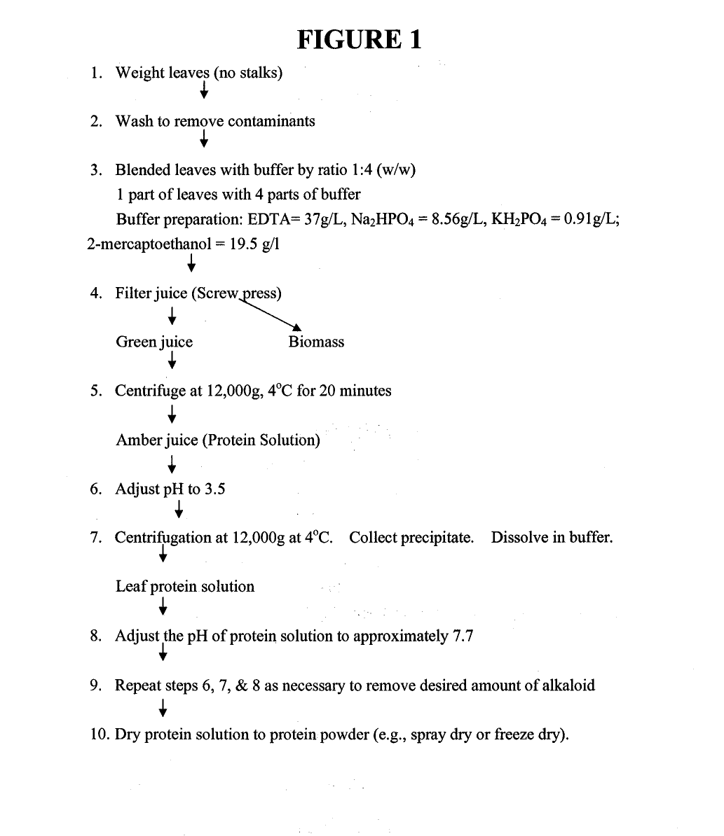

Methods of the invention may be used to isolate water-soluble leaf proteins from any plant of interest. In some embodiments, methods of the invention may be used to isolate water-soluble leaf proteins from solanaceous plants. Examples of suitable plants include , but are not limited to, tobacco, other Nicotiana species, potato, tomato, eggplant, Capsicum species, and belladonna. In one embodiment, methods of the invention may be used to isolate water-soluble leaf protein from tobacco plants. One suitable method is shown schematically in FIG. 1 and described below using tobacco plants. One skilled in the art will appreciate that analogous steps would apply in the case of other plants (e.g., solanaceous plants), such as other Nicotiana species, potatoes, or tomatoes.

This invention relates to removal of nicotine from a solution containing water-soluble leaf proteins derived from tobacco or other solonaceous plants. The manner of obtaining this initial solution is known to practitioners in the art. In one common method, as described in Wildman (1982) or Lo et al. (2008), green leaves are crushed or macerated, and then pressed, to express a “green juice” containing the water-soluble proteins. Green particulate matter, such as chloroplasts, are then typically removed from this “green juice,” as discussed in Wildman (1982), resulting in an “amber juice” (also sometimes referred to as a “brown juice”) from which both the fibrous leaf matter and green particulate matter have been removed. This amber juice contains water-soluble leaf proteins.

In one example of the preparation of amber juice, which generally followed the method described in Lo et al. (2008), freshly harvested tobacco leaves were disrupted using a hammermill while substantially simultaneously being exposed to a buffer system consisting of 0.067 M Na₂HPO₄.KH₂PO₄. Specifically, in each liter of water, the buffer contained the following: 1000 ml (tap) water, 8.052 g Na₂HPO₄, 0.911 g KH₂PO₄, 37.2 g EDTA (as a chelating agent) and 19.53 g 2-mercaptoethanol (as a reducing agent). The buffer concentration was 0.067 M, the pH was approximately 7.7, and the buffer solution was applied at a buffer to leaf ratio of 4:1 w/w.

Following solubilization of the leaf proteins, the fibrous plant material was then removed using a screwpress. The remaining solution (often referred to as a “green juice”) was refrigerated for 12 hours at 4° C. The solution was then squeezed through four layers of cheesecloth. The resulting juice was then centrifuged at 12,000 g for 20 minutes at 4° C. The clear supernatant (i.e., the “amber juice”) was then collected and kept at 4° C. prior to analysis.

The pH of the leaf protein solution should be adjusted to approximately the point where protein starts to precipitate out of the solution. In most if not all cases, this will require some downward adjustment from the original pH of the amber juice. If the pH is not adjusted sufficiently downward to the point where protein precipitation occurs, then protein recovery will be reduced. Similarly, continuing to adjust the pH downward after precipitation begins will likely lead to protein loss.

Adjustment of the pH may be accomplished by adding, stirring or mixing in (preferably continuously) acid. Any acid known to those skilled in the art may be used in the practice of the invention. A non-limiting list of acids which may be utilized for this purpose includes the following acids: phosphoric acid, sulfuric acid, citric acid, nitric acid, tartaric acid, malic acid, lactic acid, malonic acid, succinic acid, acetic acid, glutamic acid, glucaric acid, itaconic acid, levulinic acid, furnaric acid, aspartic acid, propionic acid, monopotassium citrate, or furandicarboxylic acid. In one embodiment of the invention, 75% to 85% phosphoric acid is used to adjust the pH.

As described above, the pH may be adjusted to the point where protein precipitation is observed. Typically, this will be at a pH of from about 1.5 to about 5.5. In one embodiment of the claimed invention, the pH of the solution may be adjusted to be between from about 2.5 to about 5.0. In a particularly preferred embodiment, the pH may be adjusted to approximately 3.5.

It is possible to passively permit the soluble leaf protein to continue to precipitate out of the solution. Alternatively, separation of the protein can be accelerated through centrifugation and/or filtration. In one preferred embodiment, separation is accomplished by centrifuging the solution at 12,000 g for at least one and not more than 20 minutes. Continuous flow centrifugation may be used in the practice of the present invention. Practitioners skilled in the art will recognize that adjusting the parameters or gravitational force may alter and perhaps improve the separation of the protein and nicotine, depending on the particular characteristics of the amber juice solution. Typically, separation may be carried out at a temperature not greater than 20° C. nor less than 0° C., and preferably under refrigeration conditions (between 4° C. and 10° C.). Those skilled in the art will appreciate that centrifugation or filtration or a combination thereof may give improved separation results for a given solution. Following separation, most of the water-soluble leaf protein is contained in the precipitate (or retentate), while most of the nicotine is removed in the supernatant (or filtrate or permeate). The precipitate (or retentate) is retained.

The acid separation process described in the previous paragraph may be repeated in order to reduce the alkaloid content to a desired level. In a preferred embodiment, the precipitate is dissolved in buffer and the process described herein (beginning with adjustment of the pH of the protein-containing solution) is repeated a second time. In a particularly preferred embodiment, the process described herein is repeated a third time.

If such repeated separation is performed, it is contemplated that the leaf proteins may be dissolved in a buffering solution. Possible buffering solutions include the system combinations: sodium phosphate dibasic and potassium phosphate monobasic (Na₂HPO₄.KH₂PO₄), potassium phosphate monobasic/sodium hydroxide, sodium hydroxide/citric acid, acetic acid/ammonium acetate, potassium hydroxide/potassium phosphate monobasic, citric acid/disodium phosphate, potassium phosphate monobasic/potassium phosphate, dibasic, potassium acid phthalate/sodium hydroxide, potassium carbonate/potassium tetraborate/potassium hydroxide/disodium EDTA dihydrate, Giordano's buffer, sodium acetate trihydrate/sodium chloride, tris(hydroxymethyl)aminomethane (Tris), EDTA/Tris/HCl, 2-amino-2-(hydroxymethyl)-1,3-propanediol/Tris, Tris/EDTA, ammonium chloride/ammonium hydroxide, HEPES/NaCl, imidazole, phosphate, N-morpholinopropane sulfonic acid (MOPS), N-tris(hydroxymethyl)methyl-2-aminoethane sulfonic acid (“TES”), triethanolamine, and N-tris(hydroxymethyl)-methyl-glycine (“Tricine”), or other phosphate buffers, hydrochloric acid buffers, boric acid buffers, sodium hydroxide buffers, citric acid or citrate buffers, or carbonate buffers. These are contemplated for use with tobacco but could be appropriate for other plant species as well. As someone having ordinary skill in this art would readily understand, any buffering solution which proves effective at solubilizing leaf proteins in the pH range of the solution can be used: The buffer solution may be added in a ratio of up to 10:1 v/v compared with the original volume of the leaf protein solution prior to the addition of acid as described above. The buffer may be added in a ratio of from about 1:10 to about 10:1, from about 1:5 to about 5:1, from about 1:2.5 to about 2.5:1 v/v compared with the original volume of the leaf protein solution prior to the addition of acid as described above. In one embodiment, if the leaf protein solution initially contained 100 ml by volume prior to the acid pH adjustment and separation steps, then between 10 ml and 1000 ml of buffer solution should be added to resolubilize the precipitated leaf proteins. In a preferred embodiment, a chelating agent is included in the buffering system to reduce protein denaturation and a reducing agent is added to minimize oxidation and denaturation of the proteins. In one embodiment of this invention, a 0.067 M solution of Na₂HPO₄.KH₂PO₄, and including 10 mM of EDTA and 25 mM of 2-mercaptoethanol at a pH of approximately 7.7 at a ratio of buffer to original leaf solution of 1:1 is utilized.

Following the dissolution of the leaf protein precipitate or retentate in the buffer solution, then acid may be applied to adjust the pH to the point where the protein precipitates out of solution, as described above. A separation process, which may the same or different than the separation process used in the first iteration, is then performed, and the protein then collected. The entire process of buffering and dissolution of the leaf proteins, adjustment with acid and separation of the proteins followed by collection of the proteins may be repeated until the alkaloid concentration reaches the desired level.

Once the concentration of nicotine and other alkaloids reaches the desired level, the leaf protein precipitate or retentate can then optionally be dried down using standard commercial drying techniques (e.g., freeze drying or spray drying) to form an entirely or substantially nicotine-free and alkaline free leaf protein powder.

### EXAMPLES

### Example 1

The inventors evaluated three different approaches for removing nicotine from tobacco-derived soluble leaf proteins. In each case, the evaluations utilized an “amber juice” solution comprising water-soluble leaf protein prepared according to the method of Lo, et al. (2008) WO/2008/143914. The samples were derived from Maryland tobacco variety 609LA, a low-alkalkoid variety containing 0.6 mg/g to 0.8 mg/g of nicotine.

In the first approach, acetone (10 ml, Sigma Aldrich) stored at −20° C. was added to an amber juice leaf protein solution (10 ml) with continuous stirring, until the protein started to precipitate, followed by centrifuged at 12,000×g for 10 min at 4° C. The precipitate was collected to further assay nicotine content and soluble protein content.

In the second treatment, a Prep/Scale TFF ultrafiltration module containing polysulfone membrane (100,000 Da MWCO) (Millipore, Bedford, Mass.) was employed to reduce the nicotine content of the tobacco protein solution after extraction. A MasterFlex L/S™ peristaltic pump (Cole-Parmer Instrument Co., Vernon Hills, Ill.) was used to pump the amber juice protein solution samples through the unit. The ultrafiltration flow rate and transmembrane pressure was kept at 3 L/min and 10 psi, respectively. The retentate samples were collected in a graduated cylinder to further assay nicotine content and soluble protein content.

The third method involved a three-tier buffer treatment. The pH of the protein solution was adjusted by dropwise addition of 85% phosphoric acid (Sigma Aldrich) to the amber juice protein solution (10 ml) under continuous stirring, until the solution pH reached the predetermined value of 3.5 where the protein started to precipitate. The solution was then centrifuged at 12,000×g for 10 min at 4° C. Precipitate was then collected to assay nicotine content and soluble protein content. The precipitate was then dissolved in a buffer containing 0.067 M Na₂HPO₄.KH₂PO₄ at a pH of approximately 7.7 (10 ml). Following dissolution of the precipitate, the procedure was repeated twice for a total of three repetitions of the treatment with 85% phosphoric acid. Each of the precipitate samples was collected and analyzed for nicotine content and soluble protein content.

The concentration of nicotine in the samples was determined using reverse-phase high-performance liquid chromatography (HPLC). The sample was separated by using a Shimadzu LC2010A (Columbia, Md.) equipped with serial dual plunger pumps, an oven, an automated sampling injection unit, and an ultraviolet-visual (UV-VIS) detector (D₂ lamp light source) with a wavelength range of 190 to 600 nm. A 300×3.9 mm I.D. μBondapak™ C18 column (Waters, Ireland) was used. The mobile phase was methanol-citrate phosphate buffer (15:85, v/v, apparent pH 2.4 adjusted with addition of perchloric acid) controlled at 0.7 ml/min flow rate under 35° C. column temperature. The column effluent was monitored at 260 nm with UV-Vis spectrophotometric detector. The nicotine concentration of samples was determined by the nicotine standard (Sigma Aldrich) solution (1.0 to 60 g/ml) prepared in the mobile phase. Sample injection volume was 20 μL.

Experiments to optimize tobacco protein parameters were designed by response surface methodology (RSM) using the quadratic model in SPSS software (Ver. 10.0.5, SPSS Inc., Chicago, Ill.). The number of trials and the corresponding measurements are shown in Table 1. Data is presented as the means ±standard deviation (SD) of three replicates, and Student's t-test was used to assess the significance of the data.

TABLE 1
Nicotine residue content of tobacco protein product and
soluble protein recovery with 3 different removal nicotine methods.

| | Nicotine content | Soluble protein |
|---|---|---|
| Nicotine Removal Methods | (mg/g protein) | recovery (%) |
| Tobacco protein solution | 6.26 ± 0.05^{a} | 100 |
| Extraction with −20° C. acetone | ND^{b} | 63.3 ± 3.0 |
| Ultrafiltration | 3.29 ± 0.04^{a} | 94.7 ± 2.3^{a} |
| pH 3.5 Precipitation with 85% | | |
| phosphoric acid | | |
| First precipitation | 0.37 ± 0.01^{a} | 98.1 ± 2.4^{a} |
| Second precipitation | 0.03 ± 0.002^{a} | 96.0 ± 0.6^{a} |
| Third precipitation | ND^{b} | 94.5 ± 1.0^{a} |

^{a}Mean ± S.D.; n = 3.
ND^{b} is not detected.

The HPLC data indicated that no nicotine residue remained in the soluble leaf protein precipitate after being extracted by −20° C. acetone. However, tobacco protein recovery was only 63.31% of the extracting product, due to protein denaturation. This denaturation cannot be avoided in most organic solvents, even at the low temperature of −20° C. Thus, while use of acetone at a low temperature is an efficient and highly effective method of removing nicotine from leaf proteins, it also causes substantial denaturation of the proteins.

The ultrafiltration technique yielded a protein recovery of 94.77%, but nicotine levels remained at 3.29 mg/g protein following treatment with ultrafiltration. While ultrafiltration offers the advantage of lower cost and did not substantially degrade protein structure, approximately 53% of the original nicotine content of the tobacco material retained in retention volume. Thus, we did not find ultrafiltration to be the optimal technique for nicotine removal from leaf protein.

As for phosphoric acid precipitation, the salt formed from the reaction between nicotine and phosphoric acid is dissolvable in water, and can be easily separated from the tobacco protein precipitates formed in the same acidic system in the range of protein isoelectric points. The nicotine content of tobacco protein product and protein recovery yield in systems of pH3.5 to pH 5.5 adjusted by phosphoric acid is shown as Table 2.

TABLE 2
Nicotine residue content of tobacco protein
product and soluble protein recovery in different
pH value adjusted by phosphoric acid.

| | Nicotine content | Soluble protein |
|---|---|---|
| pH | (mg/g protein) | recovery(%) |
| pH 3.5 | 0.37 ± 0.01 ^{a} | 98.12 ± 01.2 ^{a} |
| pH 4.0 | 0.49 ± 0.01 ^{a} | 77.33 ± 0.7 ^{a} |
| pH 4.5 | 0.58 ± 0.01 ^{a} | 59.39 ± 1.5 ^{a} |
| pH 5.0 | 0.66 ± 0.02 ^{a} | 51.81 ± 2.1 ^{a} |
| pH 5.5 | 0.78 ± 0.02 ^{a} | 49.68 ± 1.3 ^{a} |

^{a} Mean ± S.D.; n = 3

The data show that nicotine content decreased and protein recovery increased with the decreasing pH value. At pH3.5, nicotine 0.37 mg/g of protein and 98.12% protein recovery were achieved. The results suggested that the most nicotine-phosphate formed in the low pH. We found that even a single treatment of protein precipitation at pH 3.5 with phosphoric acid achieved over 90% nicotine removal. Following a second precipitation, only a slight amount of nicotine residue −0.03 mg/g of protein remained, and 96.04% protein recovery was achieved. Repeating this treatment a third time at pH 3.5 reduced the nicotine to non-detectable levels, with the protein recovery at 94.56%.

We found that both the acetone treatment and the three steps of pH 3.5 system precipitation with phosphoric acid were effective in removing nicotine and other alkaloids from tobacco-derived leaf proteins to non-detectable levels. We believe that the three steps of pH 3.5 system precipitation with phosphoric acid (or other comparable system) is the most preferred method, because this method also provided soluble protein recovery of nearly 95%.

### Example 2

We conducted a separate test, to measure total alkaloids using a continuous flow analyzer. The purpose of this example was to measure the content of all alkaloids in the treated samples, and not merely nicotine. We tested two treated samples. One sample had been treated using the acetone treatment, while the second sample had been treated using the triple replication of the protein precipitation with phosphoric acids. The Quantification Limit=0.01% of dry matter. The results are shown in Table 3.

TABLE 3
Total Alkaloid Analysis

| | | Phosphoric Acid |
|---|---|---|
| | Acetone | (triple replication treatment) |
| Total Alkaloids | BQL | BQL |

BQL = below quantification limit

In each case, we found the total alkaloid level to be below the quantitation limit on a 0.01% of dry matter basis. This result demonstrated that the phosphoric acid treatment (performed three times) resulted in removal of all alkaloids from the leaf protein and not merely nicotine.

### REFERENCES

Fantozzi, P., Rossi, M., Schiraldi, A., and Montanari, L., “Removal of Nicotine from Tobacco Leaf Protein by Supercritical CO₂. Ital. J. Food. Sci. 4:333-339 (1993).

Pirie, N. W., “Leaf Protein and its by-products in human and animal nutrition.” Cambridge University Press: Cambridge, U.K. pp. 15-16 (1987)

U.S. Patent Documents

| | | |
|---|---|---|
| 5,497,792 | March 1996 | Prasad, et al. |
| 5,462,072 | October 1995 | Browne, et al. |
| 4,347,324 | August, 1982 | Wildman, et al. |
| 4,289,147 | September 1981 | Wildman, et al. |
| 4,153,063 | May 1979 | Roselius, et al. |
| 4,068,671 | January 1978 | Casey |

U.S. Patent Applications

| | | |
|---|---|---|
| 20090302377 | December 2008 | Nauryzbaev, et al. |

PCT Patent Applications

| | | |
|---|---|---|
| WO/2008/143914 | May 2008 | Lo, et al. |

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

Modifications may be made without departing from the basic spirit of the present invention. Accordingly, it will be appreciated by those skilled in the art that within the scope of the appended claims, the invention may be practiced other than has been specifically described herein.

1. A method for removing alkaloids from soluble leaf proteins, comprising:
(a) applying an acid to a solution containing water-soluble leaf proteins;
(b) separating the alkaloids from the water-soluble leaf proteins; and
(c) collecting the water-soluble leaf proteins.

2. The method of claim 1, wherein acid is added until the solution has a pH of from about 2.5 to about 5.0.

3. The method of claim 2, wherein the pH of the solution is approximately 3.5.

4. The method of claim 1, wherein the acid is selected from the group consisting of phosphoric acid, sulfuric acid, citric acid, nitric acid, tartaric acid, malic acid, lactic acid, malonic acid, succinic acid, acetic acid, glutamic acid, glucaric acid, itaconic acid, levulinic acid, fumaric acid, aspartic acid, propionic acid, monopotassium citrate, or furandicarboxylic acid.

5. The method of claim 1, wherein the acid comprises phosphoric acid.

6. The method of claim 1, wherein the acid is 85% phosphoric acid.

7. The method of claim 1, further comprising dissolving the collected leaf proteins and repeating steps (a), (b), and (c).

8. The method of claim 7, further comprising dissolving the collected leaf proteins and repeating steps (a), (b), and (c).

9. The method of claim 1, wherein separating comprises centrifugation or filtration.

10. The method of claim 9, wherein separating comprises centrifugation at 12,000 g for at least one minute and not more than 20 minutes.

11. (canceled)

12. The method of claim 11, wherein the ratio of buffer solution to the solution containing water-soluble leaf proteins is between approximately 1:10 and 10:1 v/v.

13. The method of claim 12, wherein the ratio of buffer solution to the solution containing water-soluble leaf proteins is 1:1 v/v.

14. The method of claim 11, wherein the buffering solution comprises a buffer system of Na₂HPO₄ and KH₂PO₄ at a concentration of approximately 0.067 M, approximately 37 g/l of EDTA, and 19.5 g/l of 2-mercaptoethanol at a pH of approximately 7.7.

15. The method of claim 12, wherein the buffering solution comprises a buffer system of Na₂HPO4 and KH₂PO₄ at a concentration of approximately 0.067 M, approximately 37 g/l of EDTA, and 19.5 g/l of 2-mercaptoethanol at a pH of approximately 7.7.

16. The method of claim 14, wherein the buffering solution comprises chelating agent and/or a reducing agent.

17. The method of claim 15, wherein the buffering solution comprises a chelating agent and/or a reducing agent.

18. The method of claim 7, wherein dissolving the leaf proteins comprises use of a buffer solution which comprises a buffer system selected from the group consisting of sodium phosphate dibasic and potassium phosphate monobasic (Na₂HPO₄.KH₂PO₄), potassium phosphate monobasic/sodium hydroxide, sodium hydroxide/citric acid, acetic acid/ammonium acetate, potassium hydroxide/potassium phosphate monobasic, citric acid/disodium phosphate, potassium phosphate monobasic/potassium phosphate, dibasic, potassium acid phthalate/sodium hydroxide, potassium carbonate/potassium tetraborate/potassium hydroxide/disodium EDTA dihydrate, Giordano's buffer, sodium acetate trihydrate/sodium chloride, tris(hydroxymethyl)aminomethane (Tris), EDTA/Tris/HCl, 2-amino-2-(hydroxymethyl)-1,3-propanediol/Tris, Tris/EDTA, ammonium chloride/ammonium hydroxide, HEPES/NaCl, imidazole, phosphate, N-morpholinopropane sulfonic acid (MOPS), N-tris(hydroxymethyl)methyl-2-aminoethane sulfonic acid (“TES”), triethanolamine, and N-tris(hydroxymethyl)-methyl-glycine (“Tricine”).

19. The method of claim 8, wherein dissolving the leaf proteins comprises use of a buffer solution which comprises a buffer system selected from the group consisting of sodium phosphate dibasic and potassium phosphate monobasic (Na₂HPO₄.KH₂PO₄), potassium phosphate monobasic/sodium hydroxide, sodium hydroxide/citric acid, acetic acid/ammonium acetate, potassium hydroxide/potassium phosphate monobasic, citric acid/disodium phosphate, potassium phosphate monobasic/potassium phosphate, dibasic, potassium acid phthalate/sodium hydroxide, potassium carbonate/potassium tetraborate/potassium hydroxide/disodium EDTA dihydrate, Giordano's buffer, sodium acetate trihydrate/sodium chloride, tris(hydroxymethyl)aminomethane (Tris), EDTA/Tris/HCl, 2-amino-2-(hydroxymethyl)- 1,3 -propanediol/Tris, Tris/EDTA, ammonium chloride/ammonium hydroxide, HEPES/NaCl, imidazole, phosphate, N-morpholinopropane sulfonic acid (MOPS), N-tris(hydroxymethyl)methyl-2-amino ethane sulfonic acid (“TES”), triethanolamine, and N-tris(hydroxymethyl)-methyl-glycine (“Tricine”).
